(19) United States
(12) Patent Application Publication
(10) Pub. No.: US 2004/0185458 A1
(43) Pub. Date: Sep. 23, 2004

(54) Analysing device for analysing chemical and/or biological samples

(76) Inventor: Andreas Bosio, Koln (DE)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/472,069

(22) PCT Filed: Mar. 16, 2002
(86) PCT No.: PCT/EP02/02950

(30) Foreign Application Priority Data
Mar. 16, 2001 (DE) 101125453

(51) Int. Cl. C12Q 1/68; C12M 1/34

(52) U.S. Cl. 435/6; 435/287.2

(57) ABSTRACT

An analyzing device for analyzing chemical and/or biological samples comprises a plurality of base parts (12) provided in a receiving device (10), for example object supports. Said base parts are sealed by means of a head part (26,80), which is configured in such a way that each base part (12) and the head part (26,80) form a test chamber (38) for receiving the samples. The analyzing device also comprises a foot part (58,88) for receiving the base part (12). The support (58,88) and the head part (26,80) are displaceable in relation to each other in order to seal the test chamber (38). The head piece (26,80) and/or the foot part (58,88) are arranged in such a way that the base part is oriented relative to the head part (56) when the test chamber is sealed. As a result, deterioration of the base parts (12) is prevented when the test chamber (38) is closed.

The invention relates to an analyzing device for analyzing chemical and/or biological samples. The analyzing device comprises a base part such as an object support or another sample support. Further, a lid is provided so that a test chamber is formed by the base part and the lid.

With such analyzing devices, tissue samples, for example, are analyzed by means of a hybridization process. In this connection, DNA pieces, for example, i.e., gene fragments, are applied upon the surface of an object support. Applying the DNA pieces is effected by dripping by means of a robot. Thus, the position of the individual DNA pieces on the object support is known. The DNA pieces connect with the surface of the object support and adhere thereto so that their positions do not change in the subsequent analyzing process.

From a tumor to be examined, for example, RNA is taken in the next step. By means of enzymes, the RNA is transformed into DNA and subsequently marked with suitable markers, particularly fluorescent color markers.

Additionally, a comparative sample with healthy tissue is produced. The healthy DNA is also marked with a suitable marker. Preferably, the marker is a fluorescent marker of another color so that the healthy tissue is marked with a greenly fluorescent marker and the tissue to be analyzed taken from the tumor, for example, with a red color marker.

Subsequently, both samples are applied onto the entire object support. The DNA strands included in the two samples firmly connect to the counterparts, i.e., the DNA pieces present on the surface of the object support. Connecting the DNA included in the samples with the DNA pieces adhering to the object support is effected in a hybridization process. Subsequently, the object support is washed so that only firmly adhering DNA pieces and chained-up DNA from the two samples is present on the object support.

After the object support has been dried, it is put to a detecting process. Therein, the individual positions of the object support to which DNA pieces adhere are analyzed by a suitable microscope. In doing so, the individual DNA pieces are stimulated by laser light, for example, so that the fluorescent markers fluoresce in the corresponding color. If a certain position to which a DNA piece adheres appears as a red spot, for example, it can be concluded therefrom that this gene was active in the tumor tissue but not in the healthy tissue. If a spot fluoresces greenly, it can be concluded therefrom that this gene was only active in the healthy tissue. In the case of yellow fluorescence occurring, the corresponding gene was active in both tissues. By the above method, it can be diagnosed which genes are active in a tumor, for example. Therefrom, conclusions as to the kind of tissue change and the like can be drawn.

In order to realize an as good connection of the two samples with the DNA pieces on the object supports as possible, it is known to close the object support by a lid, for example, so that a test chamber is formed between the object support and the lid. Then, the test chamber is vibrated by a vibration means to effect a movement of the two samples. By this movement, it is easier for the corresponding sample components to find the suitable counterparts with which they then connect. Providing a vibration means has the disadvantage that stationary waves are produced and thus, only a limited movement of the sample occurs.

The problems described in the example above also exist in other analyses of chemical and/or biological samples in which one sample, for example, firmly adheres to a base part such as an object support and another sample is to react with it. Particularly when using object supports of glass as base part, there is the danger that they are damaged when the lid is put on or during other handling operations.

It is the object of the invention to provide an analyzing device for analyzing chemical and/or biological samples where the danger of damaging the base part, particularly an object support serving as base part, is reduced.

This object is solved by an analyzing device according to claim 1.

The analyzing device according to the invention comprises a base part and a head part or lid. The base part and the head part form a test chamber. The base part may be a flat object support, for example, made of thin glass. Likewise, the object support or another sample support may be placed upon the base part so that the sample support is arranged within the test chamber. According to the invention, the analyzing device comprises a foot part for receiving the base part. The foot part is configured such that the foot part and the head part are movable relative to each other. Thereby, the test chamber is sealed. According to the invention, the head part and/or the foot part are further supported such that the base part is able to align relative to the lid upon sealing the test chamber. Thus, the base part completely abuts on all the receiving points of the head part, a circumferential seal, for example, before the base part is pressed against the head part or lid to provide for a preferably tightly sealed test chamber. Thus, the base part is freely movable, at least within certain limits, so that the occurrence of tensions in the base part is avoided upon sealing the test chamber. Thereby, the danger of damage to the base part, which particularly is a flat object support of thin glass, is considerably reduced.

The base part is preferably flat. The portion of the head part arranged against the base part is flat as well. The foot part and/or the head part are preferably supported such that the head part and the base part align in a plane-parallel manner relative to each other upon closing the test chamber.

If it is a foot part, for example, in which the base part is held horizontally, the support of the foot part, for example, permits at least a restricted movability of the base part about a horizontal axis. Preferably, a restricted possibility of movement about both horizontal axes exists. With a head part with a preferably circumferential seal, it is thus ensured that the base part is in plane abutment on the seal upon closing the test chamber. Thus, the seal does not transfer any punctual forces to the base part but only plane forces. Thereby, damage to the base part upon closing the test chamber is avoided.

A head part configured in addition or so as to be supported instead of the foot part is preferably also configured such that it is pivotable about one, preferably both, longitudinal axes. The longitudinal axes are the two horizontal axes when the base part is arranged horizontally.

In a particularly preferred embodiment, the head part and/or the foot part are supported floatingly. This can be realized, for example, by elastic receptacles, intermediate part of the like. Preferably, elastic elements, such as elastic pins, for example, are provided for the floating support of the head part and/or the foot part. It is particularly preferred to provide springs as elastic elements. Preferably, at least three elastic elements and, particularly preferably, at least four elastic elements are provided per head part and/or per foot part. Through the floating support of the head part and/or the foot part according to the invention, the forces occurring upon sealing the test chamber are evened out so that no forces occur that are substantially higher punctually, by which a damage to or destruction of the base part can be caused.

In another particularly preferred embodiment, the foot part and/or the head part are supported by means of a single-point bearing. It is configured as a ball-and-socket joint, for example, and preferably arranged centrally with respect to the base part. Thus, a movement of the foot part and/or the head part, as described above, is made possible in a simple manner. The single-point bearing is preferably a spherical bearing in the form of a ball-and-socket joint or the like.

Further, it is possible to floatingly support the base part. To this end, a deformable, preferably elastically deformable body, for example, is provided below the base part. Preferably, it is a cuboidal elastic body on which the base part, an object support, for example, is supported. The floating support can be provided in addition to the afore-described bearing of the foot part.

Preferably, the device according to the invention comprises a mover for moving the sample in the test chamber. According to the invention, the analyzing device comprises a conveying means as a mover. By means of the conveying means, at least a part of the sample is sucked off the test chamber and subsequently supplied to the test chamber again. It is also possible to make the sample circulate, the sample then being moved at least partially “in a circle”.

By providing, according to the invention, a conveying means as mover of the sample, the occurrence of stationary waves is avoided. By sucking off and supplying a part of the sample, it is ensured that the entire sample, quantity present in the test chamber is moved.

Depending on the size of the test chamber, this can be ensured, for example, by sucking off a sufficient amount of the sample and subsequently supplying it to the test chamber again. Therefore, a receiving chamber for receiving and subsequently delivering a part of the sample is preferably provided between the conveying means which, for example, is a pump, and the test chamber. By dimensioning the receiving chamber correspondingly, a large part of the sample located in the test chamber can be sucked off and subsequently supplied to the test chamber again, for example. Instead of a separate receiving chamber, a channel located between the conveying means and the test chamber can be dimensioned correspondingly and serve as receiving chamber.

In a particularly preferred embodiment, at least two receiving chambers are provided. They are connected with one or two conveying means such that a part of the sample is alternately received and delivered by the test chambers. While sample is delivered from the one receiving chamber, for example, sample can simultaneously be received in the other test chamber. Thereby, a uniform movement of the sample in the test chamber is realized. There may also be an interval between the delivery of the sample from the one receiving chamber and the reception of the sample in the other receiving chamber. Further, the two processes may overlap.

For improving the movement of the sample in the test chamber, several channels, preferably in the head part, can be arranged through which the sample is extracted from the test chamber at different sites and supplied thereto again. The extraction site may possibly also differ from the supply site so that the sample is delivered “in a circle”.

Preferably, the test chamber is formed by a corresponding configuration of the head part, such as a circumferential frame-shaped projection provided at the head part. This has the advantage that a conventional object support, a flat glass plate, for example, can be used as a base part. The frame-shaped projection provided at the head portion preferably comprises a circumferential seal so that the test chamber is sealed to the outside and no sample liquid can escape from between the head part and the base part.

In another preferred embodiment, the test chamber is formed by a frame-shaped intermediate part. Preferably, the latter is not firmly connected with the head part but either only laid between the head part and the foot part or releasably connected with the head part or the foot part. This has the advantage that the intermediate part can be easily removed and cleaned.

For forming an extremely small test chamber, the head part has a projection partially protruding into the recess of the intermediate part in order to form the test chamber. In this connection, the intermediate part is slightly thicker than the height of the projection connected with the head part. The gap formed thereby corresponds to the test chamber. Since object supports are preferably provided as base part, the recess in the intermediate part is preferably rectangular and the projection at the head part preferably cuboidal. Preferably, the test chamber has a volume of less than 100 pl, particularly less than 80 μl and particularly preferably less than 60 μl.

Preferably, a seal is arranged between the projection provided at the head part and the intermediate part so that a tight test chamber can be formed. Preferably, the seal is connected with the intermediate part so that the seal can be easily cleaned. In case of a damaged seal, it is further simpler to exchange the intermediate part than to replace a seal provided at the projection of the head part.

In a particularly preferred embodiment, a receiving device for several object supports or base parts is provided. The receiving device comprises a bottom part and centering elements connected with the bottom part. Through the centering elements, several receiving areas for receiving one object support, respectively, are configured. Thus, one object support per receiving area can be laid onto the bottom part. Then, the object support serves as base part and forms a test chamber together with a head part. To this end, a separate head part and/or foot part per receiving area can be provided. It is also possible to provide a common head part or foot part formed correspondingly for several receiving areas.

In this embodiment, it is essential for the invention that at least the foot part or the head part is movably supported for each base part. It is thus possible to provide a common foot part for several base parts and a separate head part for each base part Further, this is possible the other way round so that a common head part and separate foot parts are provided. The separately provided head parts or foot parts which are movably supported are preferably movably held at a carrier portion. It is thus possible to also move the movably held parts in common, in a vertical direction, for example, for opening or closing the test chamber. Preferably, however, the movement is effected by the common foot part or the common head part.

Thus, one object support to which DNA pieces already adhere at predetermined positions, for example, can be laid in the receiving areas of the receiving device, respectively. Then, the receiving device carrying several object supports can be easily handled by means of robot grip arms or the like, for example. Since the receiving device can be easily gripped at the centering elements or at the bottom part, for example, handling the object support is easily possible. By the device according to the invention, it is particularly ensured that object supports of thin glass are not damaged during handling. Touching the samples already located on the object supports is avoided as well.

Preferably, the centering elements are configured like integral frame parts. Thus, each object support is surrounded by a frame. This has the advantage that the object support is disposed in the receiving area in a protected manner. Preferably, the frame part has a height higher than the thickness of the object support so that the object support is received completely within the frame part. Thereby, damaging the object support upon handling the receiving device as well as touching the surface of the object support are avoided. Preferably, several receiving areas are formed by a single frame part. In this case, each receiving area is completely surrounded by a frame. A wall of the frame part provided between two neighboring object supports thus serves as portion of the frame of two receiving areas.

Each of the above-described frame parts may surround a receiving area, preferably completely as a circumferential centering frame. But they may also be individual frame parts not surrounding the receiving areas but only serving, for example, to receive corners of the object support to center the latter in the receiving device. In this case, inserting the object supports into the receiving device is possibly made easier. For making the insertion of the object supports into the receiving device easier, the centering elements may further be chamfered. The chamfer is configured such that the receiving area in which one object support is respectively received is enlarged upwards to make the insertion of an object support easier.

In a particularly preferred embodiment, the bottom part comprises flexible material at least in the receiving areas. Particularly, the entire bottom part consists of a continuous flexible material. Thus, the flexible diaphragm serves as floating support of the base part, such as the object support, for example. Thereby, the danger of damaging the base part upon closing the test chamber is considerably reduced.

The flexible material is particularly a diathermic diaphragm. Thereby, it is possible in a particularly advantageous manner to provide a tempering means such as a Peltier element below the individual receiving areas to heat individual object supports. By means of the tempering means, a very specific heating of possibly only individual partial regions of an object support is possible without having to handle the object support directly. In this case, damaging the object support is again avoided. Preferably, the diaphragm is designed such that temperatures of 4° C. to 100° C. can be transferred to the object support. The tempering means may also be provided at or in the head part and/or the foot part independent of the provision of a flexible diaphragm.

Further, cooling elements as well as cooling ribs may be provided at the head part and/or the foot part. Thereby, the temperature prevailing in the test chamber can be set precisely.

Furthermore, a positioning means for positioning the receiving means with respect to the foot part is preferably provided. For each base part provided in the receiving device, for example, a separate foot part may be provided. By means of the positioning means, the foot part can be aligned relative to the base parts, such as object supports, arranged in the receiving device. The positioning means may be configured such that each foot part is adapted to be separately aligned with respect to the base parts. Preferably, the individual foot parts provided for the base parts are aligned in common with the positioning means.

To this end, the positioning means preferably comprises a gripping element for holding the receiving device. The positioning means may particularly be a drawer-like positioning means into which the receiving means is adapted to be inserted. The drawer-like positioning means is able to be transported out of and into an analyzing device. The drawer is configured such that it simultaneously serves as positioning means and thus determines the position between the one or more foot parts and the individual base parts.

To be able to use the receiving device according to the invention particularly in existing arrangements, the outer dimensions of the receiving device correspond to standard dimensions of microtiter plates. The receiving device has a width of 96±4 mm and a length of 128±4 mm in particular.

By means of the afore-described analyzing device, hybridization analyses of DNA fragments can be performed, for example. Further, analyses of chemical and/or biological samples having a detectable affinity to each other are possible, which may be analyses of antigen-antibody affinity in particular.

Hereinafter, the invention will be explained in detail with respect to a preferred embodiment with reference to the accompanying drawings. In the Figures:

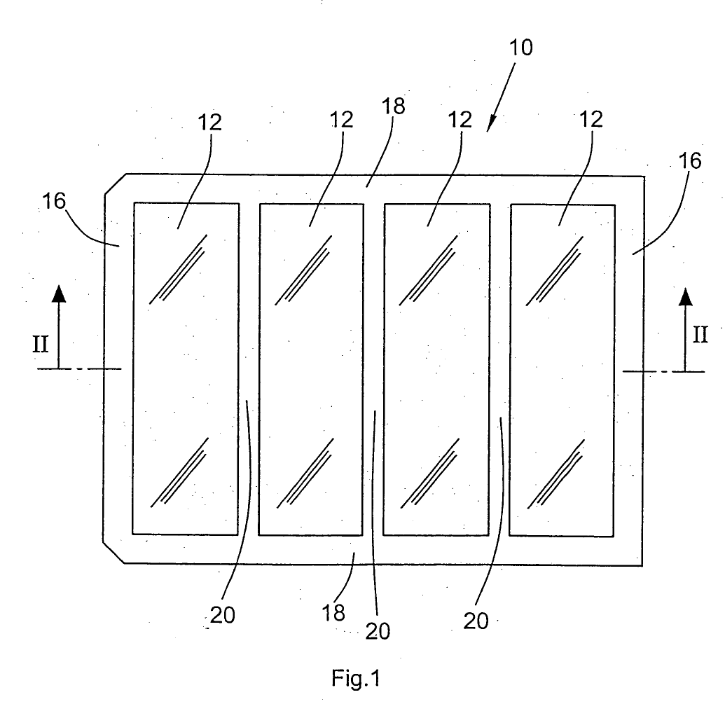

FIG. 1 is a schematic top view of a first preferred embodiment of the receiving device with several inserted base parts,

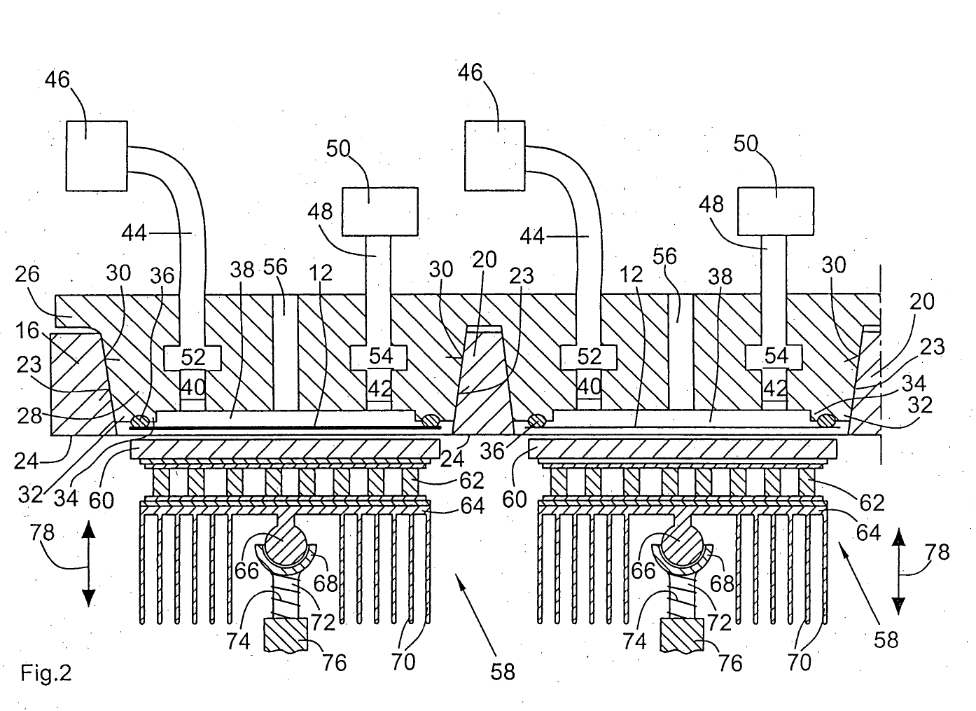

FIG. 2 is a schematic partial sectional view along the line II-II in FIG. 1, the head part not illustrated in FIG. 1 and the foot part according to the invention being additionally illustrated,

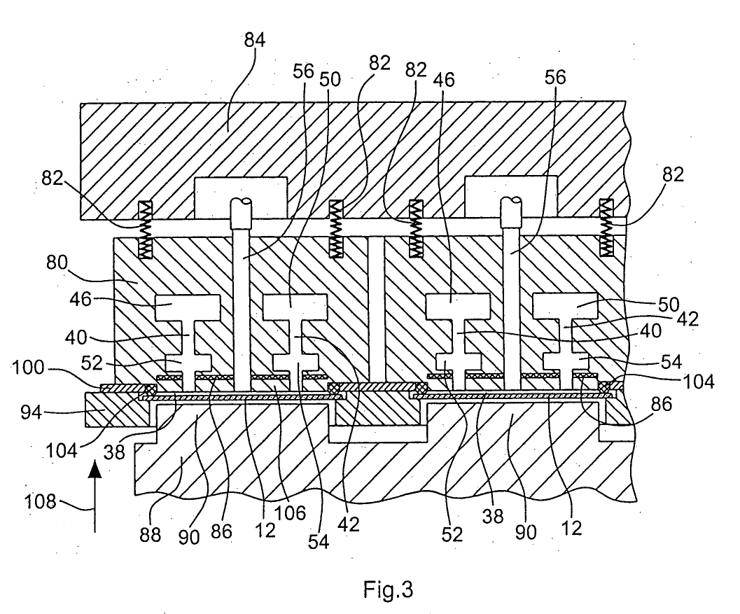

FIG. 3 is a schematic partial sectional view of a second preferred embodiment of the device according to the invention, corresponding to FIG. 2,

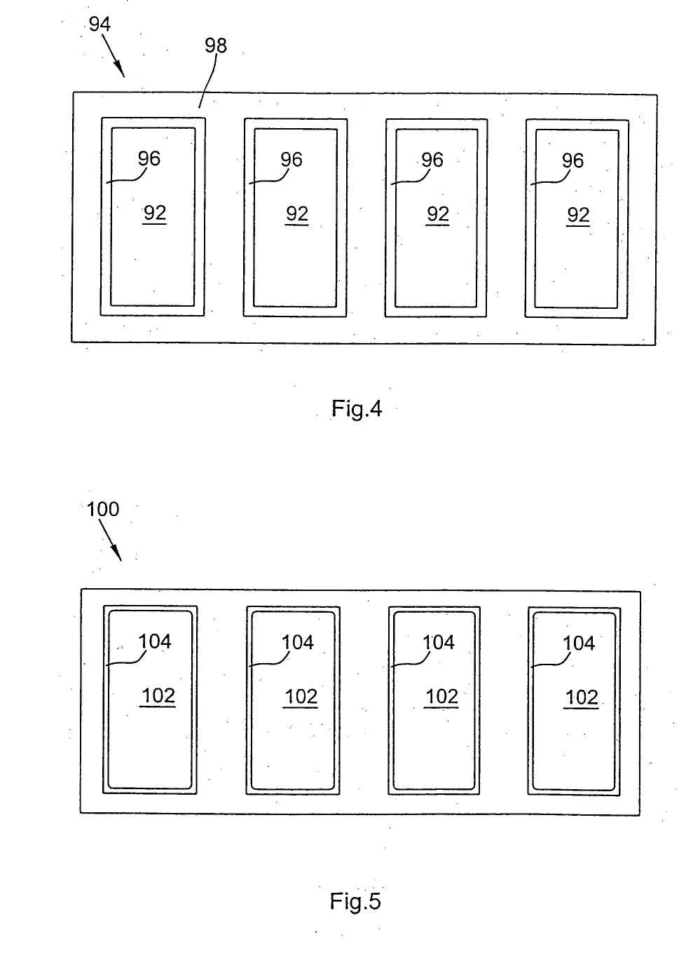

FIG. 4 is a schematic top view of a second preferred embodiment of a receiving device for base parts utilized in the device illustrated in FIG. 3,

FIG. 5 is a schematic top view of an intermediate part utilized in the embodiment illustrated in FIG. 3,

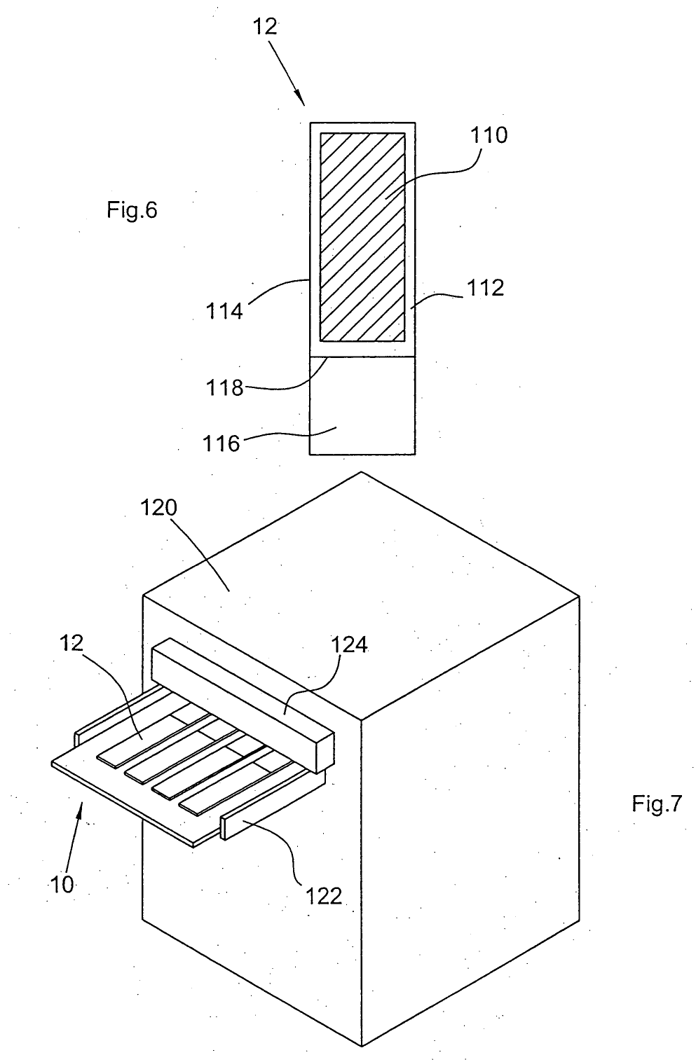

FIG. 6 shows a single base part being adapted to be inserted into the receiving device illustrated in FIG. 1, and

FIG. 7 is a schematic perspective view of an analyzing device into which the receiving device is adapted to be inserted.

A receiving device 10 for several object supports 12 comprises a bottom part 14 (FIG. 2). The bottom part 14 is connected with centering elements 16,18,20. In the illustrated embodiment, the centering elements 16,18,20 are arranged such that four rectangular receiving areas 22 are formed in each of which an object support 12 can be arranged. The centering elements 16,18,20 have two opposing shorter side walls 16 and two also opposing longer side walls 18 arranged between the side walls 16. In the illustrated embodiment, three partition walls 20 arranged in parallel to the shorter side walls 18 are provided between the side walls 18.

At the undersides 24 of the centering elements 16,18,20, the bottom part 14 configured as a diaphragm is mounted. The receiving areas 22 hence have a flexible diaphragm as a bottom on which an object support 12 is respectively supported. Thus, it is possible to press from below against the diaphragm in FIG. 2 in order to horizontally align the object support, for example. Further, a heating element can be put against the object support 12 from below. Due to the flexibility of the diaphragm, the object support 12 flatly lays against the surface of a flat heating element. Thereby, a uniform and good heat transmission between the heating element and the object support is ensured.

At their insides 23, i.e., at the sides pointing toward the receiving areas 22, the centering elements 16,18,20 are chamfered. Thereby, the opening of the individual receiving areas pointing upwards in FIG. 2 is upwardly enlarged. This makes the insertion of the object supports 12 into the receiving areas 22 easier.

A head part 26 is configured such that it projects into the individual receiving areas 22 formed between the centering elements 16,18,20. To this end, the head part 26 comprises four projections 28 in the illustrated embodiment which have a substantially rectangular cross-section. The side walls 30 of the projections 28 abut on the inner walls of the web-shaped centering elements 16,20. Likewise, side walls of the projection 28 not illustrated in FIG. 2 abut on the web-shaped centering elements 18. On the underside of the head part 26 facing the base part or sample support, frame-shaped projections 32 are provided for each receiving area 22. The frame-shaped projection is cirumferential. At an underside 34 of the frame-shaped projection 32, a seal 36 is provided which is circumferential as well. The seal 36 is supported on an outer border of the sample support 12 und seals the sample support with respect to the head part 26. Due to the frame-shaped projection 32, a test chamber 38 is formed between a head part inside 36 and the sample support 12 within the frame-like projection 32.

For moving samples provided in the test chamber 38, two channels 40,42 connected with the test chamber 38 are provided in the head part 26. The channel 40, for example, is connected to a pump 46 via a hose 44. Correspondingly, the channel 42 is connected with a pump 50 via a hose 48. The two pumps 46,50 are controlled by a preferably common control unit. The pumps 46,50 alternately suck a part of the sample located in the test chamber off into the channel 40 and 42, respectively, and subsequently deliver it into the test chamber 38 again. Thereby, a movement of the sample in the test chamber 38 is realized so that matching sample parts connect more easily if, for example, the device is used to analyze DNA affinities.

Further, two receiving chambers 52,54 are provided within the head part 26. The receiving chamber 52 is connected with the channel 40 and the receiving chamber 54 is connected with the channel 42. Due to the size of the receiving chambers 52,54, the sample quantity taken from the test chamber 38 can be collected. It is also possible that the two pumps 46,50 are directly connected with the receiving chambers 52,54 and are possibly arranged within the head part 26 or at the upper side thereof.

In order to supply a sample into the test chamber 38, a further channel 56 connected with the test chamber 38 is provided in the head part 26. After the head part 26 has already been set upon the receiving device 10, samples can be supplied through this channel 56 into the test chamber 38 which is already tightly sealed. Likewise, the sample can be supplied through one of the two channels 40,42, which is possibly branched for this purpose.

For improving the movement in the test chamber 38, the channels 40,42 may be branched so that several channels 40,42 are connected with the test chamber 38. Further, it is possible to provide a plurality of channels 40,42 per test chamber 38 in the head part 26. Preferably, half of the channels are connected with the same pump.

On the side of the receiving device 10 opposite to the head part 26, a foot part 58 is provided. The foot part 58 comprises a plane plate 60 adapted to be heated. Preferably, the heatable plate 60 is rectangular. Preferably, the outer dimensions of the heatable plate are slightly larger than the outer dimensions of the base part 12. The heatable plate 60 preferably is a metal plate or another heat-conductible plate.

In FIG. 2, a thermoelectric cooler 62 as a heating element is provided below the heatable plate. Said cooler can be used to heat or cool the plate 60 via a corresponding control.

A receiving plate 64 is connected with the cooler 62. Preferably, the receiving plate 64 has a rectangular cross-section as well. The outer dimensions of the plate 64 are slightly smaller than those of the base part 12. In the middle of the plate 64, a ball-shaped projection 66 is provided which serves as spherical bearing. The ball-shaped projection 66 is borne in a ball cup 68. Thereby, a movement of the entire foot part 58 about the two axes horizontal in FIG. 2 is possible.

In addition, cooling ribs 70 are connected with the plate 64. The cooling ribs 70 serve to permit a faster cooling of the sample liquid in the test chamber 38 after a heating process, for example.

The ball cup 68 is supported by a centrally arranged rod-shaped foot part 72. About the foot part 72, a helical spring 74 is arranged that is supported, on the one hand, at the underside of the ball cup 68 and, on the other hand, in an abutment 76 that is rod-shaped as well. By means of the spring 74, vertical impacts can be damped in FIG. 2.

In order to close and to open the test chamber 38, the foot part 58 is movable in the direction of an arrow 78. In addition to or instead of the movability of the foot part 58 in the direction of the arrow 78, the head part 26 in FIG. 2 may be configured to be vertically movable, i.e., in the direction of the arrow 78 as well.

The head part 26, for example, may be held in a non-illustrated holding device. In FIG. 2, the holding device may be vertically moved. Because of the vertical movement of the holding device together with the head part 26, an automatic lowering of the head part 26 into the receiving areas 22 of the receiving device 10 is possible. In this case, the seal 36 provided at the frame-shaped projections 32 is pressed upon a border portion of the sample supports 12 and the test chamber 38 is formed. This can be supported by a movement of the foot part 58 in the direction of the arrow 78. Since the sample supports 12 are supported on an elastic diaphragm serving as bottom part 14, damaging the sample supports by lowering the head part 26 and/or lifting the foot part 58 is avoided. Further, the elastic diaphragm 14 serves to ensure a tight sealing between the head part 26 and the sample supports 12.

A further preferred embodiment of the device according to the invention will be explained in detail hereinafter with reference to the FIGS. 3-5, similar or like components of the device being designated with the same reference numerals as those used in FIGS. 1 and 2.

The substantial difference of the device according to the invention illustrated in FIG. 3 with respect to the device according to the invention illustrated in FIG. 2 consists in that a separate head part 80 is provided for each base part 12. Via elastic elements 82, which are preferably four springs per head part 80, each head part 80 is connected with a carrier part 84. Instead of providing four springs per head part 80 as elastic elements 82, it is also possible to provide a disc spring for each head part 80. Thereby, the head parts 80 can be moved about the two longitudinal axes arranged in the horizontal plane within certain limits.

Corresponding to the embodiment illustrated in FIG. 2, channels 40,42,56, receiving chambers 52,54 as well as pumps 46,50 connected with the channels 40,42 are provided within the head part. Furthermore, a tempering means 86 is arranged within the head part 80 in the embodiment illustrated in FIG. 3. By the tempering means 86, the sample located in the test chamber 38 can be heated or cooled.

Opposite to the head parts 80, a common foot part 88 is provided. In the illustrated embodiment, the die-like foot part 88 comprises cuboidal projections 90 projecting into rectangular recesses 92 (FIG. 4) of a receiving device 94.

The receiving device 94 serves to receive the base parts or object supports 12. To this end, the receiving device 94 serving to receive four object supports 12 has a step 96. The object supports 12 are supported on the steps 96. By means of the frame-shaped portion 98 of the receiving device 94, which surrounds the steps 96, the object supports 12 are centered in the recess 92. It is, of course, also possible that the frame-shaped portion 98 of the receiving device 94 is interrupted. This has the advantage that it is more easily to insert and remove the object support 12.

Furthermore, an intermediate part 100 (FIG. 5) is arranged between the head parts 80 and the foot part 88 in addition to the receiving device 94. The intermediate part 100 also comprises four recesses 102 that are rectangular in correspondence with the recesses 92. In this connection, the recesses 102 of the intermediate part 100 are smaller than the recesses 92 of the receiving device 94. The recess 102 has a circumferential seal 104.

In order to form the test chamber 38, a preferably cuboidal projection 106 of the head part 80 engages into the recess 102. Since the projection 106 is of lesser height than the thickness of the intermediate part 100, the test chamber 38 is formed between the underside of the projection 106 and the upper side of the object support 12. In this connection, the seal 104 abuts on the upper side of the object support 12, on the one hand, and also seals with respect to the projection 106 of the head part 80, on the other hand. In order to form a tight test chamber 38, the head part 88 is moved in the direction of the arrow 108. Thereby, the projections 90 of the foot part 80 press against the underside of the object support 12 and press the latter against the seal 104.

The individual object supports 12 serve, for example, for analyzing DNA samples. To this end, each object support, as illustrated in FIG. 6, is divided into different areas. In an inner area 80, sample droplets are applied, for example, which then firmly connect to the sample support 12. Thus, the area 80 serves as surface for arranging an array. The area 80 is surrounded by a preferably 2.5 mm wide margin 82. The margin 82, for example, serves to that all the samples provided in the area 80 have a sufficient distance to the outer edges 84 of the object support 12. With the head part 26 being lowered, the seal 36 is supported in the margin area 82. An area 86 separated from the margin 82 by the line 88 illustrated in FIG. 6 serves to touch the sample support 12. In this area, no samples are disposed so that the object support 12 can be handled in the area 86. The touch area 86 is required, for example, to be able to insert the object support 12 into the receiving device 10 according to the invention.

The receiving device 10 or the receiving device 94 together with the intermediate part 100 can be inserted into an analyzing device 90 (FIG. 7) comprising an hybridization head and the head part 26, respectively. To this end, the analyzing device 90 comprises a drawer-like receiving means 92 for receiving the receiving device 10. The drawer may be configured in correspondence with a drawer of a CD player or the like. When the drawer 92 is opened, the receiving device 10 supporting the object supports 12 or the receiving device 94 together with the intermediate part 100 are inserted into the drawer from above and then displaced into the analyzing device 90.

Within the analyzing device 90, the head part 26 is arranged such that it is lowered downward upon the object supports 12 after the receiving device 10 has been displaced into the analyzing device 90.

Additionally, a drying head 94 is provided above the drawer 92. The drying head 94 is provided with a fan. After the hybridization process has been completed, the drawer is displaced out of the analyzing device 90 again. In doing so, the sample or washing liquid remaining on the object supports 12 is removed by means of the drying head 94. Preferably, the drying head has several outlet and inlet openings. By the inlet openings, the air delivered by the drying head or another drying medium is sucked in again. Thereby, it is avoided that sample liquid components reach the surroundings. In this connection, the cross-sectional area of the suction inlet is larger than that of the outlet so that the drying head 94 always sucks in more air than it delivers. Thereby, the danger of components of the sample reaching the surroundings is further reduced. The inlets and/or outlets may be slot-shaped openings extending over the width of the drying head.

To ensure a complete drying of the sample supports 12, the drawer 92 may be displaced back and forth several times.

Subsequently, the receiving device 10 or 94, possibly together with the intermediate part 100, is removed from the drawer again.

Additionally, it is possible to wash the sample supports before drying them. Preferably, this is done by means of the head part 26,i.e., when the test chambers 38 are still sealed. Thus, the washing procedure is performed within the analyzing device. To this end, a washing liquid can be supplied via separate channels provided in the head part 26 or via the channels 40,42 of the test chamber and drained therefrom again. Before the head part is displaced upwards within the analyzing device and the test chamber is no longer tight therefor, the liquid in the test chamber is preferably sucked off to a large extent. Sucking off, in turn, is preferably effected via the channels 40,42 already present.

1. An analyzing device for analyzing chemical and/or biological samples, comprising
a test chamber (38) for receiving the sample, formed by a base part (12) and a head part (26,80), and
a foot part (58,88) for receiving the base part (12), the foot part (58,88) and the head part (26,80) being movable relative to each other for closing the test chamber (38),
characterized in
that the foot part (58,88) and/or the head part (26,80) are supported such that the base part (12) aligns itself relative to the head part (26,80) upon closing the test chamber (38).

2. The analyzing device according to claim 1, characterized in that the bearing is configured such that the head part (26,80) and the base part (12) align relative to each other in a plane-parallel manner;

3. The analyzing device according to claim 1 or 2, characterized in that the head part and/or the foot part are supported so as to be pivotable about a longitudinal axis and/or a transverse axis.

4. The analyzing device according to one of claims 1-3, characterized in that the head part (26,80) and/or the foot part (58,88) are floatingly supported.

5. The analyzing device according to one of claims 1-4, characterized in that the bearing is effected by at least one elastic element (82).

6. The analyzing device according to claim 5, characterized in that at least three, preferably at least four elastic elements are provided per head part (80) and/or per foot part.

7. The analyzing device according to one of claims 1-3, characterized in that the foot part (58,88) and/or the head part (26,80) are supported by a single-point bearing (66,68).

8. The analyzing device according to claim 7, characterized in that the single-point bearing (66,68) is a spherical bearing.

9. The analyzing device according to one of claims 1-8, characterized in that the base part (12) is floatingly supported.

10. The analyzing device according to one of claims 1-9, characterized in that the test chamber (38) is formed by a circumferential frame-shaped projection (32) provided at the head part (26).

11. The analyzing device according to claim 10, characterized in that the projection (32) comprises a seal (36).

12. The analyzing device according to one of claims 1-9, characterized in that the test chamber (38) is formed by a frame-shaped intermediate part (100).

13. The analyzing device according to claim 12, characterized in that the head part (88) comprises a preferably cuboidal projection (106) partially projecting into the recess (102) of the intermediate part (100) for forming the test chamber (38).

14. The analyzing device according to claim 13, characterized in that a seal (104) is provided between the projection (106) and the intermediate part (100).

15. The analyzing device according to one of claims 1-14, characterized in that the base part is a flat sample support (12).

16. The analyzing device according to one of claims 1-15, characterized in that the head part (26,80) comprises a supply channel (56) for supplying the sample into the test chamber.

17. The analyzing device according to one of claims 1-15, characterized by a receiving device for several base parts (12), comprising
a bottom part (14), and
centering elements (16,18,20;98) connected with the bottom part (14), the centering elements (16,18,20;98) being arranged such that several receiving areas (22) are configured to receive one base part (12), respectively.

18. The analyzing device according to claim 17, characterized in that the centering elements (16,18,20;98) are formed by integral frame parts and the frame part is formed such that each receiving area (22) is surrounded by a circumferential centering frame (16,18,20;98).

19. The analyzing device according to claim 17 or 18, characterized in that the centering elements (16,18,20;98) are chamfered in the direction of the receiving area (22).

20. The analyzing device according to one of claims 17-19, characterized in that the bottom portion (14) consists of flexible material, particularly of a diathermic flexible diaphragm, at least in the receiving areas (22).

21. The analyzing device according to one of claims 17-20, characterized in that the intermediate part (100) is arranged between the receiving device (94) and the head part (80).

22. The analyzing device according to one of claims 17-21, characterized in that the plurality of base parts (12) is received by a common foot part (88) or a common head part (26) and a separate head part (80) or a separate foot part (58) is provided per base part (12).

23. The analyzing device according to one of claims 1-22, characterized in that several head parts (80) and/or foot parts are movably held at a carrier portion (84).

24. The analyzing device according to one of claims 1-23, characterized by a mover for moving the sample in the test chamber.

25. The analyzing device according to claim 24, characterized in that the mover comprises a conveying means (46,50) for sucking off a part of the sample from the test chamber (38) and supply it to the test chamber (38) again.

26. The analyzing device according to claim 24 or 25, characterized in that the conveying means is connected with a receiving chamber (52,54) for receiving and subsequently delivering a part of the sample.

27. The analyzing device according to claim 26, characterized in that at least two receiving chambers (52,54) are provided that alternately receive and deliver a part of the sample.

28. The analyzing device according to claim 26 or 27, characterized in that at least one receiving chamber (52,54) is provided in the head part (26).

29. The analyzing device according to one of claims 1-28, characterized in that the head part (26,80) and/or the foot part (58,88) comprise a tempering means (60,62,64;86).

30. The analyzing device according to one of claims 17-29, characterized by a positioning means (92) for positioning the receiving device (10) with respect to the foot part (58).

31. The analyzing device according to claim 30, characterized in that the positioning means (92) comprises gripping elements for holding the receiving device (10).

32. The analyzing device according to claim 30 or 31, characterized in that the receiving device (10) is adapted to be transported into and out of the analyzing device (90) by means of the positioning means (92) in a drawer-like manner.

33. The analyzing device according to one of claims 17-32, characterized in that a drying means (94) comprising a fan is provided in the travel path of the receiving device (10).

34. The analyzing device according to claim 33, characterized in that the drying means (94) additionally comprises a sucking means.

35. The analyzing device according to claim 34, characterized in that the cross-section of an opening of the sucking means has a larger cross-section than that of an opening of the fan.
